(19) United States
(12) Patent Application Publication
(10) Pub. No.: US 2013/0345813 A1
(43) Pub. Date: Dec. 26, 2013

(54) Dual Anchor Lateral Vertebral Body Fixation Plates

(71) Applicants: Sheryl Frank, Lakeville, MA (US); John Riley Hawkins, Cumberland, RI (US); Nicholas Pavento, North Attleboro, MA (US); Jonathan Bellas, Raynham, MA (US); Ernest Quintanilha, Norton, MA (US); Michael J. O'Neil, West Barnstable, MA (US)

(72) Inventors: Sheryl Frank, Lakeville, MA (US); John Riley Hawkins, Cumberland, RI (US); Nicholas Pavento, North Attleboro, MA (US); Jonathan Bellas, Raynham, MA (US); Ernest Quintanilha, Norton, MA (US); Michael J. O'Neil, West Barnstable, MA (US)

(21) Appl. No.: 13/530,483

(22) Filed: Jun. 22, 2012

(51) Int. Cl. A61F 2/44

(52) U.S. Cl. 623/17.16

(57) ABSTRACT

Plate-based fixation system having dual anchors. The plate can include cams. The plates can have a banana-shape. The plates can be adapted to receive a polyaxial bone screw. The plates can be jointed so as to be height-adjustable.

### BACKGROUND OF THE INVENTION

A lateral access approach is frequently selected to deliver interbody fusion cages to the lumbar spine. In comparison to conventional anterior or posterior approaches to the lumbar spine, the lateral approach is thought to minimize posterior and/or anterior tissue damage as well as reduce surgery time, associated blood loss, vascular damage and infection risk.

In general, it is known in the art to mount a lateral fusion cage with a plate that secures the cage to the sides of adjacent vertebral bodies.

U.S. Pat. No. 7,594,931 (Louis) discloses an intervertebral arthrodesis implant for insertion in an intervertebral space separating opposite faces of two adjacent vertebrae. The implant has a ring-shaped intervertebral cage having a bar that extends perpendicular to the axis of the spine. The bar has a height less than the rest of the cage. A surface of the cage contacting the vertebrae has an undulating shape for limiting sliding of the cage in a plane parallel to the vertebral faces.

PCT Published Patent Application WO2011-080535 (Dinville) discloses anchoring devices, anchoring systems for intervertebral implants, intervertebral implants, and instruments and methods for implanting the implants. In preferred configurations, these various objects share the feature of comprising or cooperating with an anchoring device having a body comprising at least one curved plate elongated along a longitudinal axis. The plate is designed to be inserted through a passage crossing at least a part of the implant in order to penetrate into at least one vertebral endplate and attach this implant onto this vertebral endplate by means of at least one stop retaining the implant. The body of the anchoring device comprises at least one longitudinal rib on at least a part of at least one of its faces, the rib being designed to cooperate with a groove made in a passage of implant.

In one type of intervertebral device suited for the lateral approach, the fusion cage is mounted with a plate that secures the cage to the adjacent vertebral bodies. In particular, US Published Patent Application 2010-0004747 (Lin) discloses a spinal fixation device comprising a trans-vertebral and intra-vertebral plate and a rectangular cage with a slot for the plate for neutralizing intervertebral movement in spinal interbody fusion. The rectangular cage with a vertical or oblique slot is inserted into the intervertebral space from the lateral or anterior side of the spinal column. The plate is then inserted through the slot of the cage and hammered into and buried inside the two adjacent vertebral bodies to achieve three-dimensional intervertebral fixation.

U.S. Pat. No. 7,341,590 (Ferree) discloses an anterior thoracic/lumbar system comprising a thin plate and fasteners for securing the plate to vertebrae or other osseous material. The plate may be hinged along the central axis, with a pair of collinear holes on each portion of the plate. Each of the holes accommodates a bolt which is screwed into the vertebrae and secured to the plate using a nut.

WO 2009-025841 (Nuvasive) discloses a surgical fixation system having an improved mechanism to prevent the back out of screws employed in securing a surgical fixation plate to an intended orthopedic location.

US 2011-0213421 (Binder) discloses an apparatus for reducing the profile of a bone fixation plate while, preventing backing out of screws is disclosed. The apparatus includes at least one section of relief and sections of engagement. The plate has at least two openings though which two screws can pass through bony tissue. As the screw is tightened, it will begin to lag the plate to the bone. When the screw head interferes with the plate at the interference point, there is a slight resistance that insertion forces can overcome. When the screw is advanced further, it snaps into the sliding fit area and is allowed to move freely. The forces that cause the screw to back out from the plate are preferably not strong enough to pass the screw head back past the interference section. It may be desirable to include a set screw to help prevent backout.

US 2007-0233118 (McLain) discloses a method of implanting a fusion plate, having at least two primary fastener openings, into a patient. A throat of the patient is dissected, providing access through the throat dissection to a spinal column of the patient. The fusion plate is inserted into the throat dissection, and the fusion plate is then positioned in an asymmetrical relationship with a sagittal plane of the spinal column. A first primary fastener is inserted through a first primary fastener opening of the fusion plate and into the first vertebra. A second primary fastener is inserted through a second primary fastener opening of the fusion plate and into the second vertebra. A cervical fusion apparatus is also disclosed.

U.S. Pat. No. 8,007,523 (Wagner) discloses a spinal plate system and method for fixation of the human spine is provided. In an embodiment, the system includes a bone plate, a bone screw and a ring. The bone screw preferably connects the bone plate to a bone, and the ring preferably fixes the bone screw into a borehole of the bone plate such that the bone screw extends from the bone plate at a selected angle. The ring is preferably capable of swiveling within the borehole to allow the bone screw to be angulated at a plurality of angles oblique to the plate. The bone screw may have a head having a tapered, threaded surface for engaging the ring. The ring preferably has threading on its inner surface for mating with the threading on the head. The inner surface of the ring may be tapered. Movement of the head through the ring preferably expands the ring against the bone plate to fix the bone screw at a selected angle relative to the bone plate.

U.S. Pat. No. 8,002,808 (Morrison) discloses a device for supporting and/or assisting in bone fusion, particularly in the spine. A plate member is provided, along with two or more attachment members that are anchorable to bones. In one embodiment, the plate member has a slot near one end and an aperture at another end. The attachment members include threaded posts for connection to the plate member via the latter's slot(s) and aperture(s). Alternatively, attachment members need not have a threaded post, and attachment members may be connected to the plate member via a bone bolt or similar fixation member. The slot(s) allow a single plate member to be used for a variety of operative situations and anatomies. A device for repositioning bones and a method for using the disclosed devices is also described.

### SUMMARY OF THE INVENTION

The present invention is concerned with providing improved plating for interbody fusion cages inserted into the disc space through a lateral approach.

In accordance with the present invention, there is provided a intervertebral fusion device comprising:
a) an intervertebral fusion cage having an anterior wall, a posterior wall, leading and trailing walls connecting the anterior and posterior walls to form a central vertical throughhole, the trailing wall having a threaded insertion hole, an upper surface adapted for gripping an upper endplate and a lower surface adapted for gripping a lower endplate;
b) a bone plate comprising a bone-facing surface, an outer surface, and upper, lower and central holes passing from the outer surface to the bone-facing surface,
c) first and second bone anchors, each anchor comprising an intermediate head, a distal threaded shaft and a proximal threaded shaft, wherein the proximal shaft of each bone anchor is respectively received in one of the upper and lower holes of the bone plate so that the head of each bone anchor bears against the bone-facing surface of the plate,
d) a linear connector passing through the central hole of the plate and the insertion hole of the cage, the linear connector having a threaded distal end portion,
e) first and second threaded nuts,
f) first and second washers,
   wherein each threaded nut is threadably received on the proximal shaft of each bone anchor so that each bone plate is interposed between the threaded nut and the head of the bone anchor.
   wherein each washer is received on the proximal shaft of the bone anchor so that the washer is interposed between the respective washer and the bone plate.

Also in accordance with the present invention, there is provided a bone fixation system comprising:
a) a bone plate having a thickness, a bone contacting surface and an outer surface, and comprising:
   i) first and second screw through holes having a first threadform,
   ii) first and second recesses,
wherein each recess overlaps with a respective screw throughole,

b) first and second bone screws, each screw having a head and a shaft having a thread thereon that mates with the first threadform;
c) first and second anti-backout features,
   wherein each screw is received in a respective screw through hole and extends therethrough,

wherein each anti-backout feature is received in a respective recess,

wherein each anti-backout feature bears against the head of a respective screw, and

wherein the axial length of each cam thread is substantially the same as the axial length of its respective second threadform.

Also in accordance with the present invention, there is provided an intervertebral fusion device comprising:
a) an intervertebral fusion cage having an anterior wall, a posterior wall, leading and trailing walls connecting the anterior and posterior walls to form a central vertical throughhole, the trailing wall having a threaded throughhole, an upper surface adapted for gripping an upper endplate and a lower surface adapted for gripping a lower endplate;
b) a bone plate comprising a width, a bone-contacting surface, an outer surface, and upper, lower and central holes passing from the outer surface to the bone-contacting surface, wherein the bone-contacting surface has an upper ledge and a lower ledge extending therefrom, each ledge adapted for contacting a respective cortical rim of a vertebral body,
c) first and second bone anchors respectively received in the upper and lower holes of the bone plate,
   wherein the trailing wall of the cage is located between the upper and lower ledges.

Also in accordance with the present invention there is provided a bone fixation system comprising:

a) a bone plate having first and second throughholes therein,

b) first and second annular inserts, each annular insert comprising:
i) a distal side comprising a plurality of snap-connectors, and
ii) a proximal side comprising a concave portion of a spherical surface,

c) first and second polyaxial screws, each screw comprising a spherical screw head, wherein each plurality of snap-connectors snaps into and secures to a respective throughhole, wherein each spherical screw head is received in the concave portion of the spherical surface of each insert.

Also in accordance with the present invention, there is provided (claim 14) a bone fixation system comprising:
a) a bone plate comprising first and second halves, each half having a thickness and comprising:
   i) a bone screw through hole,
   ii) a recess having a threadform, wherein each recess overlaps with a respective screw throughhole,
   iii) a receiving portion having a receiver adapted to receive a set screw,
b) first and second bone screws, each screw having a head and a shaft having a thread thereon that mates with the first threadform;
c) first and second anti-backout feature,
d) a set screw,
wherein the receivers are aligned, and the set screw is received in the aligned receivers to join the halves of the bone plate,
wherein each bone screw is received in a respective bone screw through hole and extends therethrough,
wherein each anti-backout feature is received in a respective recess,
wherein each anti-backout feature bears against the head of a respective bone screw.

### (Recessed Hole)

Also in accordance with the present invention, there is provided (claim 30) an assembly comprising:
a) bone plate comprising a bone-facing surface, an outer surface, and upper and lower holes passing from the outer surface to the bone-facing surface, wherein the each of the upper and lower holes is at least partially surrounded by a recess extending inwards from the outer surface;
c) a pair of bone anchors, each bone anchor comprising a shaft and a head;
   wherein the shaft of each bone anchor is received in one of the respective upper and lower holes.

### DESCRIPTION OF THE FIGURES

FIG. 1 discloses a plate system of the present invention comprising a plate, a pair of nutes, a pair of washers and a pair of uniaxial screw.

FIGS. 2-3 disclose the plate system of FIG. 1 fixed to adjacent vertebral bodies along with a cage inserted within a disc space.

FIG. 4 discloses a plate system and cage within a functional spinal unit along with a retractor.

FIG. 5-6 disclose various views of a plate system having cams and a cage within a functional spinal unit.

FIG. 7 discloses a banana-shaped plate system having cams.

FIG. 8 discloses a plate having cams with a pair of ledges on the bone-contacting side of the plate.

FIGS. 9-10 the plate of FIG. 8 along with a cage within a functional spinal unit.

FIG. 11 discloses the system of FIGS. 9-10 with a retractor.

FIGS. 12-14 discloses a plate adapted to accept a polyaxial acrew.

FIG. 15 discloses the plate of FIGS. 12-14 along with a cage within a functional spinal unit.

FIG. 16 discloses a low profile plate system.

FIGS. 17-18 discloses a fixation system having a jointed height-adjustable plate.

### DETAILED DESCRIPTION OF THE INVENTION

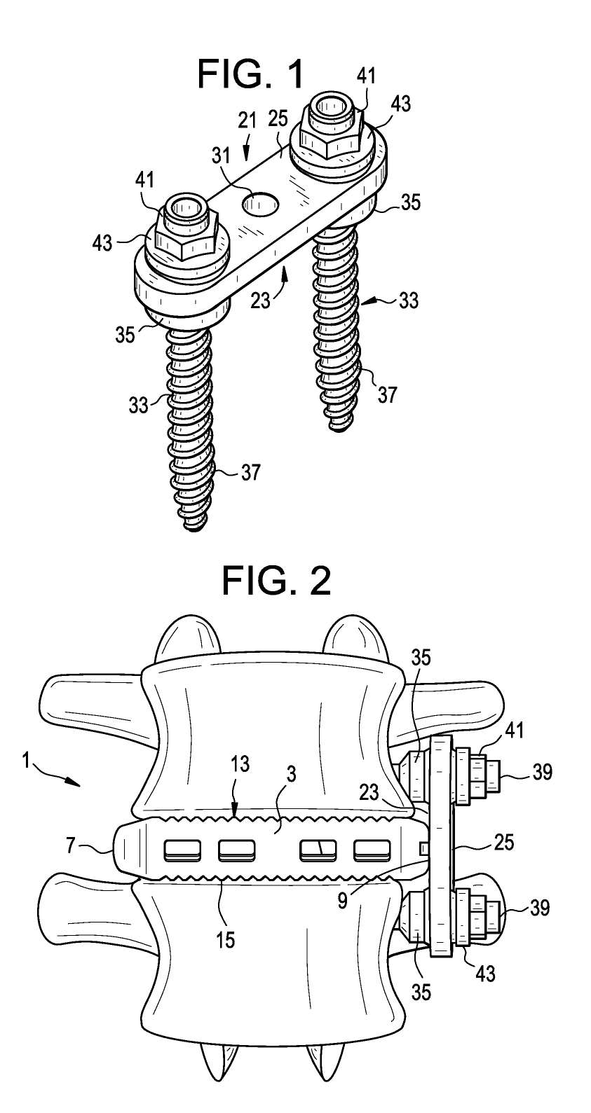

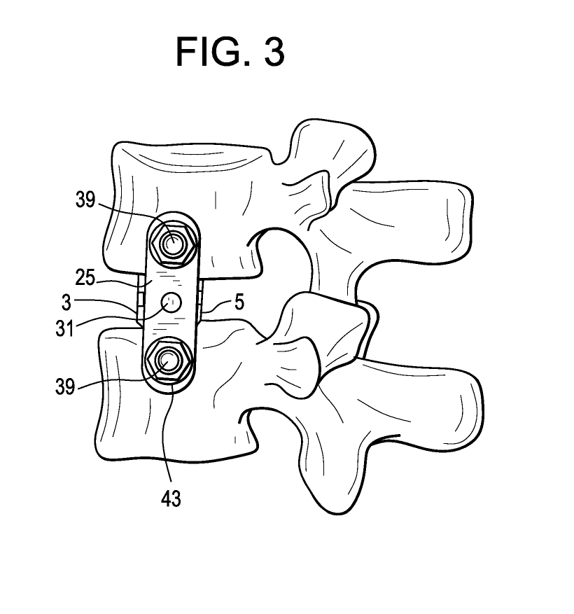

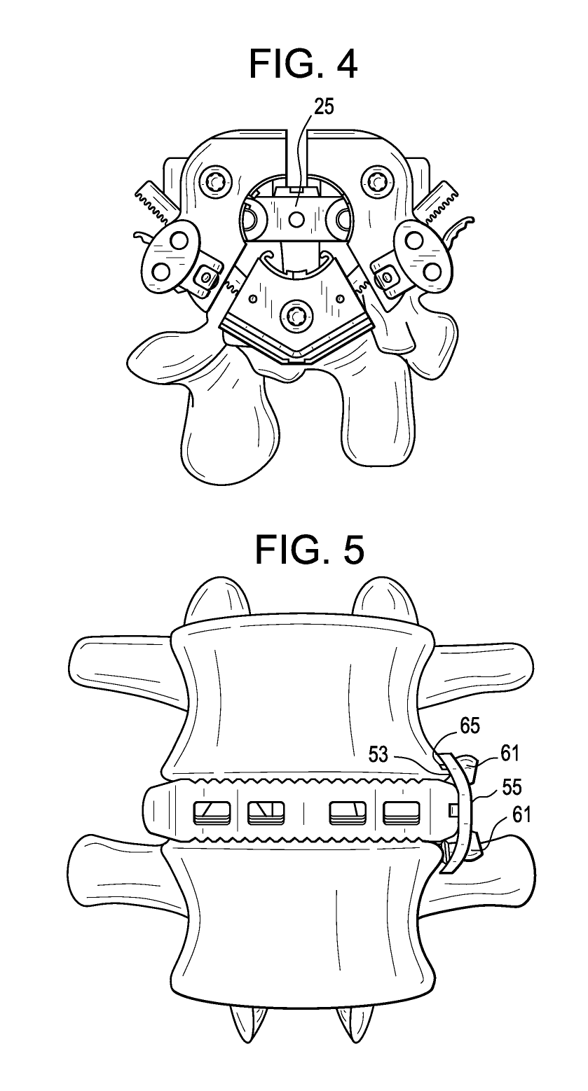

Now referring to FIGS. 1-4, one advantage of this first embodiment lies in its small width. As shown in FIG. 3, the width of the plate may be no greater than the width of the associated cage. Because the width of the plate is no greater than the width of the cage, the plate may be passed down the same MIS portal as the cage. There is no need to provide a larger portal due to the plate.

Another advantage of the first embodiment is that is can be made from conventional spinal surgery components that provide a strong, rigid construct. This may be desirable for surgeons who are already familiar and comfortable with rigid bolt-and-plate constructs.

The central hole of the plate of the first embodiment provides another advantage not found in conventional plates. After the lateral cage has been inserted, a linear connector can be passed through this central hole and connected to the insertion hole of the cage. The connector can then act as a guide to hold the plate during insertion and translate the plate to a location abutting the cage.

This first embodiment also provides a number of additional advantages. First, as it can be assembled through the use of conventional bolts nuts and washers, there is a high familiarity for the user which makes for a quick learning curve, a good safety level and simplified customer adoption. Second, because only existing components are used, the tighteners and drivers used to assemble these components can also be selected from existing off-the-shelf instruments. This reduces inventory needs. Third, there is a potential to attach the plate directly to the cage with the central hole, which matches up with the threaded hole in the rear of the cage. Fourth, there is a potential in this embodiment to use either polyaxial bolts with washers or fixed bolts without washers. The use of fixed bolts without washers provides a lower profile design. In some embodiments, the bolts may have a 5 mm, 6 mm, 7 mm, 8 mm, or 9 mm proximal/distal shaft diameter. In some embodiments, the bolts may have a length of between 30 mm and 50 mm. Lastly, this embodiment also allows for cortical rim screw fixation When a bone anchor is fixated through a vertebral body's endplate cortical rim it purchases within a thicker area of cortical bone as compared to the VB's wall and enhances the bio-mechanical strength of the finished plate/screw construct.

Therefore, in accordance with the present invention, there is provided a intervertebral fusion device comprising:
a) an intervertebral fusion cage 1 having an anterior wall 3, a posterior wall 5, leading 7 and trailing 9 walls connecting the anterior and posterior walls to form a central vertical throughhole, the trailing wall having a threaded insertion hole, an upper surface 13 adapted for gripping an upper endplate and a lower surface 15 adapted for gripping a lower endplate;
b) a bone plate 21 comprising a bone-facing surface 23, an outer surface 25, and upper, lower and central 31 holes passing from the outer surface to the bone-facing surface,
c) first and second bone anchors 33, each anchor comprising an intermediate head 35, a distal threaded shaft 37 and a proximal threaded shaft 39, wherein the proximal shaft of each bone anchor is respectively received in one of the upper and lower holes of the bone plate so that the head of each bone anchor bears against the bone-facing surface of the plate,
d) a linear connector (not shown) passing through the central hole of the plate and the insertion hole of the cage, the linear connector having a threaded distal end portion,
d) first and second threaded nuts 41,
e) first and second washers 43,
   wherein each threaded nut is threadably received on the proximal shaft of each bone anchor so that each bone plate is interposed between the threaded nut and the head of the bone anchor.
   wherein each washer is received on the proximal shaft of the bone anchor so that the washer is interposed between the respective washer and the bone plate.

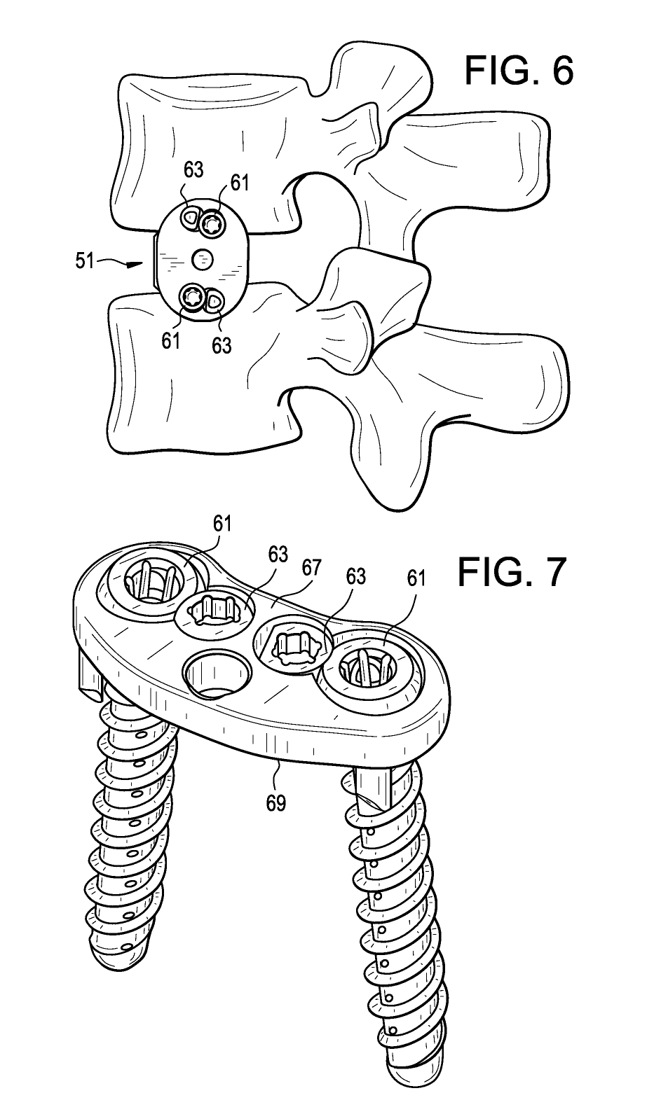

Now referring to FIGS. 5-7, the primary advantage of this second embodiment lies in its use of cams. By using cams to prevent screw backout, the surgeon can eliminate the need for nuts and washers that undesirably increase the profile of the system. Therefore, this embodiment possesses the potential to attain a very low profile. In addition, it is understood that surgeons favor plate systems in which the screws are first implanted then tightened.

Therefore, in accordance with the present invention, there is provided (claim 6) a bone fixation system comprising:
a) a bone plate 51 having a thickness, a bone contacting surface 53 and an outer surface 55, and comprising:
   i) first and second screw through holes having a first threadform,
   ii) first and second recesses having a second threadform,

wherein each recess overlaps with a respective screw throughole,
b) first and second bone screws 61, each screw having a head and a shaft having a thread thereon that mates with the first threadform;
c) first and second cams 63, each cam having a thread thereon that mates with the second threadform,

wherein each screw is received in a respective screw through hole and extends therethrough,

wherein each cam is received in a respective recess,

wherein each cam bears against the head of a respective screw, and

wherein the axial length of each cam thread is substantially the same as the axial length of its respective second threadform.

In addition, FIG. 5 also discloses a plurality of teeth 65 extending from the bone contacting surface of the plate. These teeth assist in the temporary fixation of the plate to the vertebral bodies during insertion.

In addition, the plate of FIG. 5 also has a concave shape on the bone-contacting surface of the plate. This desirably matches the convexity of the portions of the vertebral bodies to which the plate attaches.

This cam-containing embodiment also provides a number of additional advantages. First, as it can be assembled through the use of conventional screws (i.e., the AEGIS™ screw, marketed by DePuy Spine Inc. of Raynham, Mass.). Second, the tighteners and drivers used to assemble these components can also be selected from existing off-the-shelf instruments. Third, the camming mechanism can be similar to that found in the Tri-Lobe Cam-Loc™ mechanism found in the AEGIS™ plate system, marketed by DePuy Spine of Raynham, Mass. Fourth, because it uses cams, this embodiment has a much lower profile than the two bolt/hole plate of the first embodiment. Fifth, this embodiment also allows for cortical rim screw fixation. When a bone anchor is fixated through a vertebral body's endplate cortical rim it purchases within a thicker area of cortical bone as compared to the VB's wall and enhances the bio-mechanical strength of the finished plate/screw construct. Sixth, because the cams and the screws have the same drive feature, a single screwdriver may be used to tighten the screws and the cams, thereby providing the advantage of improved procedural flow and ergonomics with fewer instrument passes. Seventh, because the spherical screw head and matching cam provides for polyaxial fixation, the screw trajectories can loosely follow the path of the chamfers on the rear of the cage, thereby providing the advantage of divergent screw fixation and cortical rim fixation, both of which enhance biomechanic stability. Eighth, because of the cams ability to work at any angular dimension with respect to screw head center, this embodiment can have a nearly circular plate, thereby providing the advantage of minimizing the plate profile and thereby minimizing soft tissue stretch or damage due to retraction. In some embodiments, the plate is substantially circular. Ninth, a trial embodying the dimensions of a plate may be used to assess the suitability of different plates sizes. Tenth, conventional instruments such as a lateral plate holder and a midline plate holder may be used during the insertion of this embodiment. Eleventh, because the conventional insertion technique for the AEGIS™ screw includes anterolateral approach, it is likely that an anterolateral approach could likewise be selected for inserting this embodiment. Twelfth, in some embodiments, temporary fixation pins may be used to hold the plate in place. These fixation pins may be either threaded or unthreaded.

Now turning to FIG. 7, when the cams are oriented on the same anterior or posterior side of their associated screws, the plate can be conveniently tailored to remove extraneous metal and produce a banana-shaped plate. For the purposes of the present invention, a banana-shaped plate has a concave surface 67 and a substantially parallel convex surface 69. These banana plates have metal in the areas where they are needed (i.e., around the screw and cams, and in the region connecting the two screw-cam regions) but no more. Therefore, this design advantageously decreases the bulk of the plate and so likely can be passed down a smaller access portal (such as a retractor). In addition, in some embodiments, a banana-shaped plate can be oriented upon the vertebral bodies so that is shape essentially matches the lordotic shape of the patient's lumbar spine. In some embodiments, each cam is placed on an anterior side of its respective screw. In other embodiments, each cam is placed on a posterior side of its respective screw. In some embodiment, the cams are closer to one another than their respective screws. In some embodiment, the cams are one the same side of the screws and are closer to one another than their respective screws.

### How to use FIGS. 5-7:

After successfully accessing, clearing and sizing the disc space, select the corresponding implant, fill the cage implant with autogenous bone graft material and attach to the inserter. Gently impact the cage implant into the disc space while monitoring placement under AP fluoroscopy. Ideal placement of the implant is to support the endplate medial/laterally to the contra-lateral rim and between the anterior third and middle third of the disc space from an anterior/posterior perspective.

Select the appropriate cammed plate implant based on morphology, cage size and disc height then attach it to its respective inserter. Advancing down the access portal, insert the plate until the bone contact surface portion abuts the vertebral body rims and the ipsilateral walls of the superior and inferior vertebral bodies. Alternatively, a guide member in the insertion hole of the cage can be used to aid in advancing the plate. The plate can be slightly repositioned anteriorly or posteriorly in order to optimize the screw location or to account for anomalies such as osteophytes and vertebral body asymmetry.

Drill and Tap if necessary through the anchor holes and attach the plate with the appropriate anchors, advancing until the spherical heads are securely within their respective recesses. Turn the cams to lock the anchors in position and close the wound.

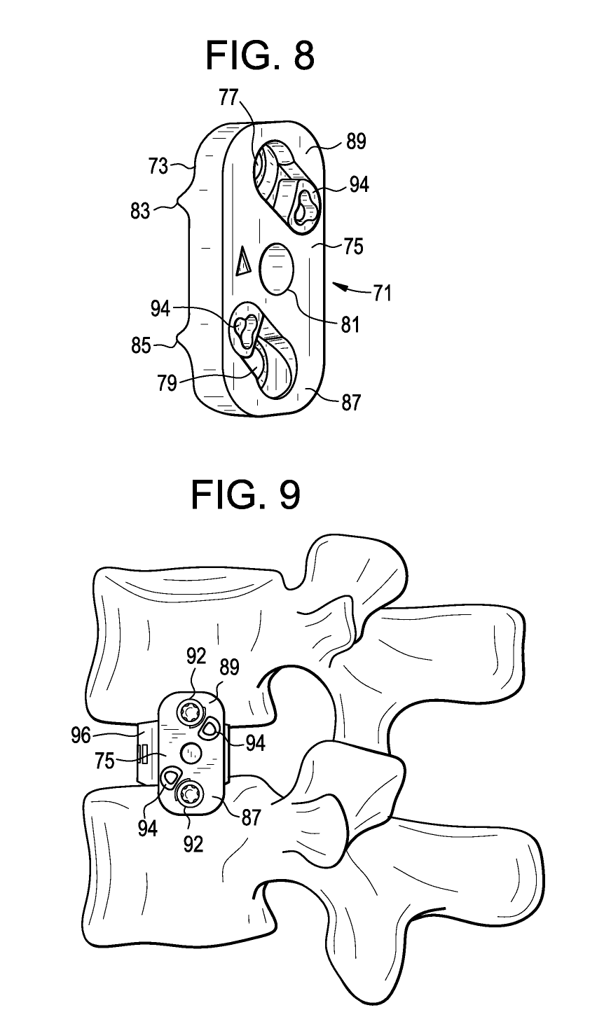

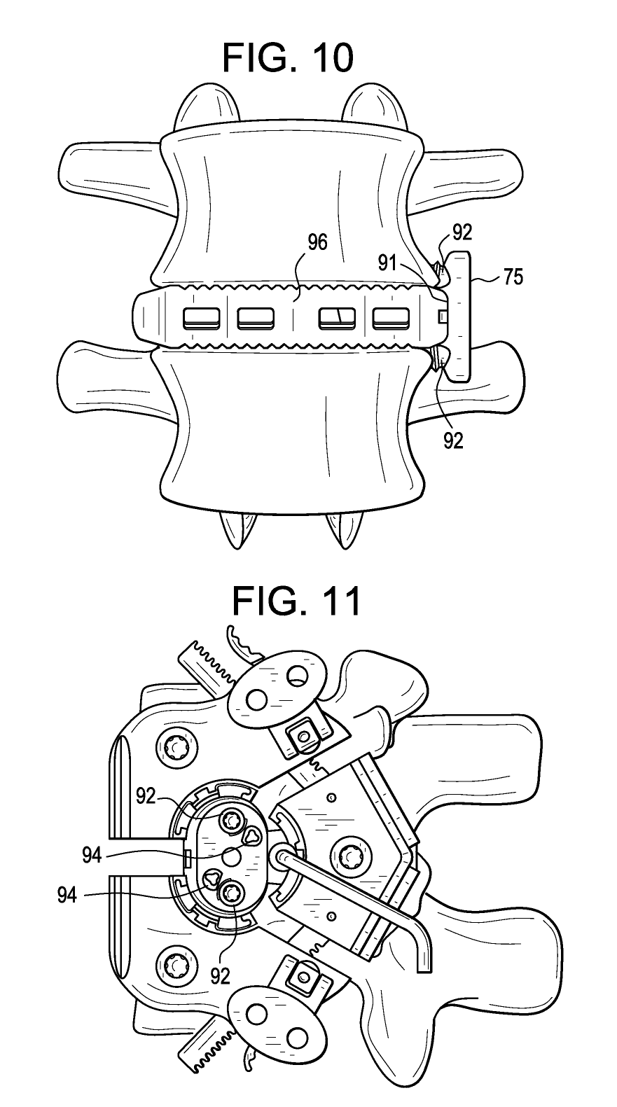

Referring now to the fixation system of FIGS. 8-11, the primary advantage of this third embodiment plate lies in its pair of ledges extending distally from the bone contacting surfaces of the plate. These ledges fill as much space as possible between the cage and the vertebral bodies, thereby providing a biomechanical advantage of stability.

Therefore, in accordance with the present invention, there is provided an intervertebral fusion device comprising:
a) an intervertebral fusion cage 96 having an anterior wall, a posterior wall, leading and trailing walls connecting the anterior and posterior walls to form a central vertical throughhole, the trailing wall having a threaded throughhole, an upper surface adapted for gripping an upper endplate and a lower surface adapted for gripping a lower endplate;
b) a bone plate 71 comprising a width, a bone-contacting surface 73, an outer surface 75, and upper 77, lower 79 and central 81 holes passing from the outer surface to the bone-contacting surface, wherein the bone-contacting surface has an upper ledge 83 and a lower ledge 85 extending therefrom, each ledge adapted for contacting a respective cortical rim of a vertebral body,
c) first and second bone anchors respectively received in the upper and lower holes of the bone plate,
   wherein the trailing wall of the cage is located between the upper and lower ledges.

In preferred embodiments, the lower ledge extends from the bone-contacting surface on a lower portion 87 of the plate and the upper ledge extends from the bone-contacting surface on an upper portion 89 of the plate.

In preferred embodiments, the ledge extends for at least 60% of the width of the plate.

In preferred embodiments, the upper and lower ledges are substantially parallel.

In some of these embodiments, the plate sits flush on the trailing wall 91 of the cage.

In some embodiments, these plates are secured by screws 92 held in place by cams 94.

This third embodiment also provides a number of additional advantages. First, as it can be assembled through the use of conventional screws (i.e., the AEGIS™ screw, marketed by DePuy Spine of Raynham, Mass.), there is no need to design and achieve regulatory clearance for any new screw. Second, the tighteners and drivers used to assemble these components can also be selected from existing off-the-shelf instruments. Third, the camming mechanism can be similar to that found in the Tri-Lobe Cam-Loc™ mechanism found in the AEGIS™ plate system, marketed by DePuySynthes Spine of Raynham, Mass. Fourth, because it uses cams, this embodiment has a much lower profile than the two bolt/hole plate of the first embodiment. Fifth, this embodiment also allows for cortical rim screw fixation When a bone anchor is fixated through a vertebral body's endplate cortical rim it purchases within a thicker area of cortical bone as compared to the VB's wall and enhances the bio-mechanical strength of the finished plate/screw construct. Sixth, because the cams and the screws have the same drive feature, a single screwdriver may be used to tighten the screws and the cams, thereby providing the advantage of improved procedural flow and ergonomics with fewer instrument passes. Seventh, because the spherical screw head and matching cam provides for polyaxial fixation, the screw trajectories can loosely follow the path of the chamfers on the rear of the cage, thereby providing the advantage of divergent screw fixation and cortical rim fixation, both of which enhance biomechanic stability. Eighth, because of the cams ability to work at any angular dimension with respect to screw head center, this embodiment can have a nearly circular plate, thereby providing the advantage of minimizing the plate profile and thereby minimizing soft tissue stretch or damage due to retraction. In some embodiments, the plate is substantially circular. Ninth, conventional instruments such as a lateral plate holder and a midline plate holder may be used during the insertion of this embodiment. Tenth, because the conventional insertion technique for the AEGIS™ screw includes anterolateral approach, it is likely that an anterolateral approach could likewise be selected for inserting this embodiment. Eleventh, in some embodiments, temporary fixation pins may be used to hold the plate in place. These fixation pins may be either threaded or unthreaded.

### How to use FIGS. 8-11:

After successfully accessing, clearing and sizing the disc space, select the corresponding implant, fill the cage implant with autogenous bone graft material and attach to the inserter. Gently impact the cage implant into the disc space while monitoring placement under AP fluoroscopy. Ideal placement of the implant is to support the endplate medial/laterally to the contra-lateral rim and between the anterior third and middle third of the disc space from an anterior/posterior perspective.

Select the appropriate plate with ledges implant based on morphology, cage size and disc height then attach it to its respective inserter. Advancing down the access portal, insert the plate until the bone contact surface portion abuts the vertebral body rims and the ipsilateral walls of the superior and inferior vertebral bodies. The plate can be slightly repositioned anteriorly or posteriorly in order to optimize the screw location or to account for anomalies such as osteophytes and vertebral body asymmetry. The ledges and contours of the bone contacting surface should intimately match the vertebral body contours. Select the plate that best fits; contouring the VB rim with an impactor or a shaping tool can be performed.

Drill and Tap if necessary through the anchor holes and attach the plate with the appropriate anchors, advancing until the spherical heads are securely within their respective recesses. Turn the cams to lock the anchors in position and close the wound.

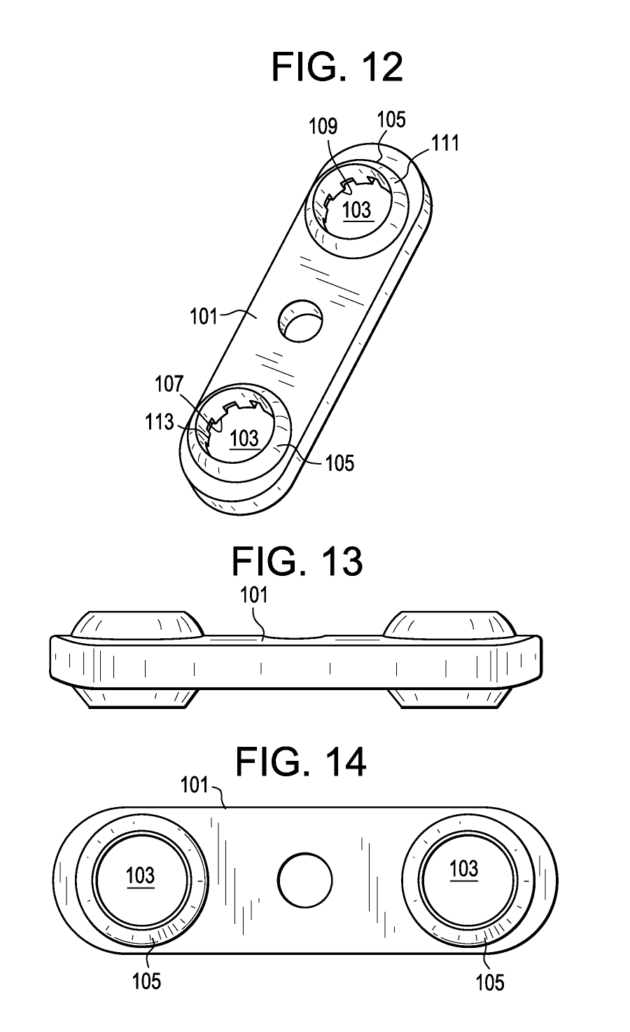

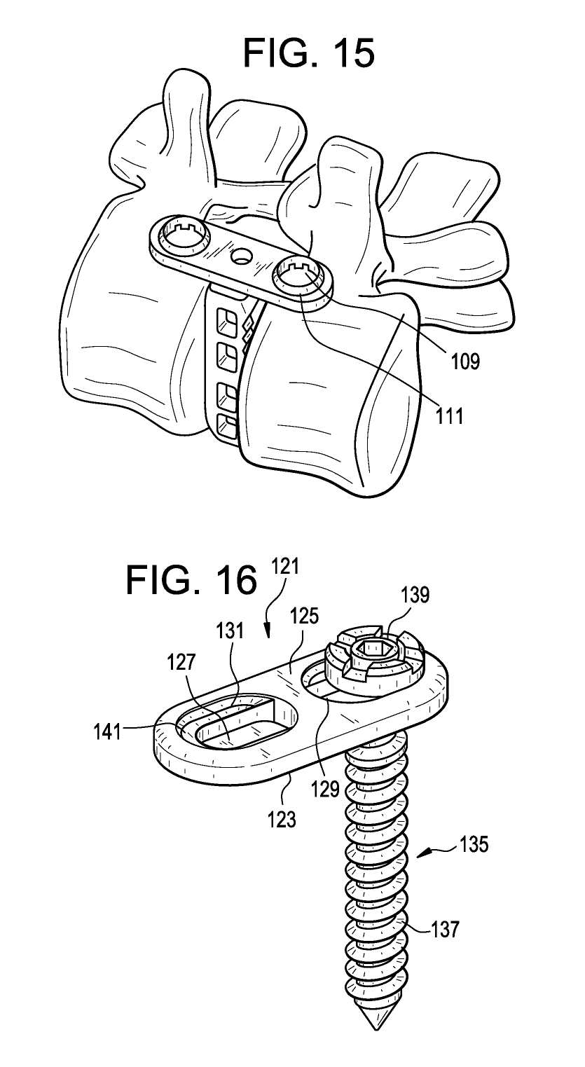

Now referring to FIGS. 12-15, there is provided an adapter for converting a conventional plate that typically receives only a conventional uniaxial bolt into a plate that can then receive a polyaxial screw.

The fourth embodiment plate of this invention is a conventional long, thin flat metal piece. Preferably, it has has an elongated throughhole that allows the bone anchor it receives to float along a single axis. The adapter (or “insert”) is preferably in the form of an annulus. The annulus comprises a) a distal side comprising a plurality of snap-connectors that allow the annulus to be snapped into and secured to the throughhole, and b) a proximal side comprising a concave portion of a spherical surface. This spherical surface receives the spherical head of a polyaxial screw. Accordingly, the spherical surface allows the use of a polyaxial bone anchor, thereby providing the surgeon with the ability to select the angle of bone anchor fixation into the vertebral bodies. This adapter may be advantageously used when it is desired to have screws that are not parallel to each other. Preferably, this adapter may be advantageously used when it is desired to have screws that are diverging from each other. It is believed that plates having divergent screws are preferred for lateral plates. Divergent or convergent screw fixation enhances the biomechanical fixation of an implant because at any given moment, expulsion forces or loosening loads generally occur in a single direction, the forces can not match both trajectories It is further believed that the spherical surface may also act as a stop against backout of the polyaxial screw.

Therefore, in accordance with the present invention there is provided (claim 13) a bone fixation system comprising:

a) a bone plate 101 having first and second throughholes 103 therein,

b) first and second annular inserts 105, each annular insert comprising:
i) a distal side 107 comprising a plurality of snap-connectors 109, and
ii) a proximal side 111 comprising a concave portion 113 of a spherical surface,

d) first and second polyaxial screws, each screw comprising a spherical screw head,

wherein each plurality of snap-connectors snaps into and secures to a respective throughhole,
wherein each spherical screw head is received in the concave portion of the spherical surface of each insert.

The FIG. 16 system represents a lower profile version of FIGS. 1-4. In this embodiment, each of the upper and lower holes is at least partially surrounded by a recess extending inwards from the outer surface, and the head of the screw is received in the recess so that the head may articulate against the recess' ledge. Therefore, this embodiment essentially has a built up portion of the plate in the area surrounding the screw head that makes for a more stiff plate.

Therefore, in accordance with the present invention, there is provided an assembly comprising:
a) bone plate 121 comprising a bone-facing surface 123, an outer surface 125, and upper 127 and lower 129 holes passing from the outer surface to the bone-facing surface, wherein the each of the upper and lower holes is at least partially surrounded by a recess 131 extending inwards from the outer surface;
b) a pair of bone anchors 135, each bone anchor comprising a shaft 137 and a head 139;
   wherein the shaft of each bone anchor is received in one of the respective upper and lower holes.

Preferably, each hole is elongated. Preferably, each of the upper and lower holes is only partially surrounded by the respective recess extending inwards from the outer surface. Preferably, the recess terminates in an inner ledge 141, and wherein the head of the screw bears against the inner ledge. Preferably, the head of the screw does not bear against the outer surface of the plate. Preferably, the head of the screw is received in the recess.

### How to use FIGS. 1-4,12-15 and 16:

After successfully accessing, clearing and sizing the disc space, select the corresponding implant, fill the cage implant with autogenous bone graft material and attach to the inserter. Gently impact the cage implant into the disc space while monitoring placement under AP fluoroscopy. Ideal placement of the implant is to support the endplate medial/laterally to the contra-lateral rim and between the anterior third and middle third of the disc space from an anterior/posterior perspective.

Select the appropriate Plate with Polyaxial insert, Expedium Offset, Blue Rings, or Elongated hole implant based on morphology, cage size and disc height then attach it to its respective inserter. Drill and Tap a pair of vertically spaced holes at the dimension of the plate holes symmetrically about the disc space. Deliver the anchors into the holes advancing until they are at equal depths. Place spherical washer on proximal portion of anchors. Advancing down the access portal, insert the plate until the anchor receiving holes capture the proximal portion of the anchors. Deliver locking washers and nut to each anchor. Tighten definitively and close the wound.

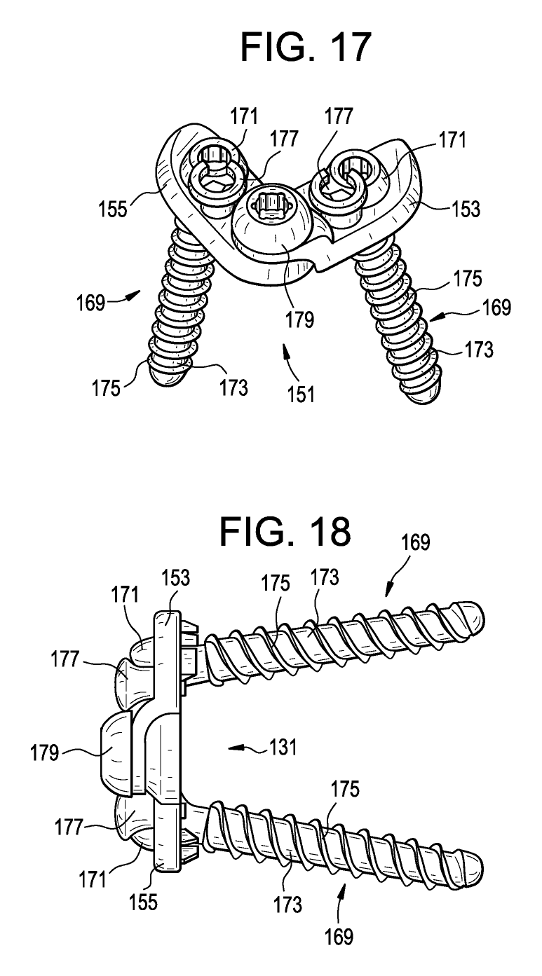

Now referring to FIGS. 17-18, in the fifth embodiment of the present invention, there is provided a bone fixation system wherein the plate is jointed. The joint in the plate allows the surgeon to intraoperatively adjust the end-to-end axial length L of the plate, thereby allowing the surgeon to use the same “one size fits all” plate on cases having different disc space heights.

Therefore, in accordance with the present invention, there is provided a bone fixation system comprising:
a) a bone plate 151 comprising first 153 and second 155 halves, each half having a thickness and comprising:
   i) a bone screw through hole,
   ii) a recess (preferably, having a threadform), wherein each recess overlaps with a respective screw throughhole,
   iii) a receiving portion having a receiver adapted to receive a set screw,
b) first and second bone screws 169, each screw having a head 171 and a shaft 173 having a thread 175 thereon that mates with the first threadform;
c) first and second cams 177,
d) a set screw 179,
   wherein the receivers are aligned, and the set screw is received in the aligned receivers to join the halves of the bone plate,
   wherein each bone screw is received in a respective bone screw through hole and extends therethrough,
   wherein each cam is received in a respective recess,
   wherein each cam bears against the head of a respective bone screw.

### How to use FIGS. 17-18:

After successfully accessing, clearing and sizing the disc space, select the corresponding implant, fill the cage implant with autogenous bone graft material and attach to the inserter. Gently impact the cage implant into the disc space while monitoring placement under AP fluoroscopy. Ideal placement of the implant is to support the endplate medial/laterally to the contra-lateral rim and between the anterior third and middle third of the disc space from an anterior/posterior perspective.

Select the jointed plate implant and attach it to its inserter. Advancing down the access portal, insert the plate until the bone contact surface portion abuts the vertebral body rims and the ipsilateral walls of the superior and inferior vertebral bodies. The plate can be slightly repositioned anteriorly or posteriorly in order to optimize the screw location or to account for anomalies such as osteophytes and vertebral body asymmetry. The plate can be re-positioned with the angle facing anterior or posterior. The plate halves can be opened or closed to adjust for optimum boney alignment. Temporary fixation pins can be used through an anchor hole or the plate can be gently impacted to allow bone contact surface features such as tynes, spikes, or ingrowth features to provisionally hold the plate's position.

Drill and Tap if necessary through the anchor holes and attach the plate with the appropriate anchors, advancing until the spherical heads are securely within their respective recesses. Turn the cams to lock the anchors in position and close the wound.

We claim:

1. An intervertebral fusion device comprising:
a) an intervertebral fusion cage having an anterior wall, a posterior wall, leading and trailing walls connecting the anterior and posterior walls to form a central vertical throughhole, the trailing wall having a threaded insertion hole, an upper surface adapted for gripping an upper endplate and a lower surface adapted for gripping a lower endplate;
b) a bone plate comprising a bone-facing surface, an outer surface, and upper, lower and central holes passing from the outer surface to the bone-facing surface,
c) first and second bone anchors, each anchor comprising an intermediate head, a distal threaded shaft and a proximal threaded shaft, wherein the proximal shaft of each bone anchor is respectively received in one of the upper and lower holes of the bone plate so that the head of each bone anchor bears against the bone-facing surface of the plate,
d) a substantially linear connector passing through the central hole of the plate and the insertion hole of the cage.

2. The device of claim 1 further comprising:
e) first and second threaded nuts,
wherein each threaded nut is threadably received on the proximal shaft of each bone anchor so that each bone plate is interposed between the threaded nut and the head of the bone anchor.

3. The device of claim 2 further comprising:
f) first and second washers,
wherein each washer is received on the proximal shaft of the bone anchor so that the washer is interposed between the respective washer and the bone plate.

4. The device of claim 1 wherein the connector has a threaded distal end portion.

5. The system of claim 1 wherein the cage has a thickness and the plate has a width, and the width of the plate is no greater than the thickness of the cage.

6. A bone fixation system comprising:
a) a bone plate having a thickness, a bone contacting surface and an outer surface, and comprising:
b) first and second screw through holes having a first threadform,
c) first and second recesses,
wherein each recess overlaps with a respective screw throughole,
d) first and second bone screws, each screw having a head and a shaft having a thread thereon that mates with the first threadform;
e) first and second anti-backout features,
wherein each screw is received in a respective screw through hole and extends therethrough,
wherein each anti-backout feature is received in a respective recess,
wherein each anti-backout feature bears against the head of a respective screw,
wherein the axial length of each cam thread is substantially the same as the axial length of its respective second threadform.

7. The device of claim 6 wherein the bone plate has a length and a width defining a length/width aspect ratio of less than 2:1.

8. The device of claim 6 wherein the bone plate has a length and a width defining a length/width aspect ratio of less than 1.5:1.

9. The device of claim 6 wherein the first cam is substantially anterior the first screw and the second cam is substantially posterior the second screw.

10. The device of claim 6 wherein a first screw is on an anterior portion of the plate and a second screw is on a posterior portion of the plate.

11. The device of claim 6 further comprising a plurality of teeth extending from the bone contacting surface of the plate.

12. The device of claim 6 wherein the bone contacting surface of the plate has a concave shape.

13. The device of claim 6 wherein each cams is placed on the same side of its respective screw.

14. The device of claim 13 wherein the plate has a banana shape.

15. The device of claim 6 wherein each cams is placed on an anterior side of its respective screw.

16. The device of claim 15 wherein the plate has a banana shape.

17. The device of claim 6 wherein each cams is placed on a posterior side of its respective screw.

18. The device of claim 17 wherein the plate has a banana shape.

19. An intervertebral fusion device comprising:
a) an intervertebral fusion cage having an anterior wall, a posterior wall, leading and trailing walls connecting the anterior and posterior walls to form a central vertical throughhole, the trailing wall having a threaded throughhole, an upper surface adapted for gripping an upper endplate and a lower surface adapted for gripping a lower endplate;
b) a bone plate comprising a width, a bone-contacting surface, an outer surface, and upper, lower and central holes passing from the outer surface to the bone-contacting surface, wherein the bone-contacting surface has an upper ledge and a lower ledge extending therefrom, each ledge adapted for contacting a respective cortical rim of a vertebral body,
c) first and second bone anchors respectively received in the upper and lower holes of the bone plate,
wherein the trailing wall of the cage is located between the upper and lower ledges.

20. The system of claim 19 wherein the upper and lower ledges are substantially parallel.

21. The system of claim 19 wherein each ledge extends for at least 60% of the width of the plate.

22. A bone fixation system comprising:
a) a bone plate having first and second throughholes therein,
b) first and second annular inserts, each annular insert comprising:
i) a distal side comprising a plurality of snap-connectors, and
ii) a proximal side comprising a concave portion of a spherical surface,
c) first and second polyaxial screws, each screw comprising a spherical screw head, wherein each plurality of snap-connectors snaps into and secures to a respective throughhole, wherein the spherical screw head is received in the concave portion of the spherical surface of each insert.

23. The system of claim 22 wherein the throughholes are elongated.

24. A bone plate comprising:
a) a bone plate having first and second throughholes therein,
b) first and second annular inserts, each annular insert comprising:
i) a distal side comprising a plurality of snap-connectors, and
ii) a proximal side comprising a concave portion of a spherical surface,
wherein each plurality of snap-connectors snaps into and secures to a respective throughhole.

25. The plate of claim 24 wherein the throughholes are elongated.

26. An assembly comprising:
a) bone plate comprising a bone-facing surface, an outer surface, and upper and lower holes passing from the outer surface to the bone-facing surface, wherein the each of the upper and lower holes is at least partially surrounded by a recess extending inwards from the outer surface;
b) a pair of bone anchors, each bone anchor comprising a shaft and a head;
wherein the shaft of each bone anchor is received in one of the respective upper and lower holes.

27. The assembly of claim 26 wherein each hole is elongated.

28. The assembly of claim 26 wherein the each of the upper and lower holes is only partially surrounded by the respective recess extending inwards from the outer surface.

29. The assembly of claim 26 wherein the recess terminates in an inner ledge, and wherein the head of the screw bears against the inner ledge.

30. The assembly of claim 26 wherein the head of the screw does not bear against the outer surface of the plate.

31. The assembly of claim 26 wherein the head of the screw is received in the recess.

32. A bone fixation system comprising:
a) a bone plate comprising first and second halves, each half having a thickness and comprising:
b) a bone screw through hole,
c) a recess having a threadform, wherein each recess overlaps with a respective screw throughhole,
d) a receiving portion having a receiver adapted to receive a set screw,
e) first and second bone screws, each screw having a head and a shaft;
f) first and second anti-backout feature,
g) a joint screw,
wherein the receivers are concentrically aligned, and the joint screw is received in the aligned receivers to join the halves of the bone plate,
wherein each bone screw is received in a respective bone screw through hole and extends therethrough,
wherein each anti-backout feature is received in a respective recess,
wherein each anti-backout feature bears against the head of a respective bone screw.

33. The system of claim 32 wherein each half of the bone plate has a bone contacting surface, and the bone contacting surface of the first half has a greater area than the bone contact surface of the second half.

34. The system of claim 33 wherein the bone contacting surface of each half of the bone plate is substantially planar, and the receiving portion extends out of the plane of the bone contacting surface of the second half of the bone plate.

35. The system of claim 32 wherein the first and second halves of the bone plate form an angle μ that is less than 180 degrees.

36. The system of claim 35 wherein the first and second halves of the bone plate form an angle μ that is at least 90 degrees.

37. The system of claim 32 wherein the bone contacting surface of each half of the bone plate is substantially planar, and the bone contacting surface of the first half is adaptable to be or not to be coplanar with the bone contacting surface of the second half

38. The system of claim 32 wherein each half of the bone plate has a bone contacting surface that is substantially planar, and each bone screw extends from its respective bone contacting surface at an angle γ that is less than 90 degrees.

39. The system of claim 32 wherein each anti-backout feature is a cam.
